(19) United States
(12) Patent Application Publication
(10) Pub. No.: US 2011/0223124 A1
(43) Pub. Date: Sep. 15, 2011

(54) HAIR CARE COMPOSITIONS AND METHODS

(76) Inventors: Tatiana V. Drovetskaya, Basking Ridge, NJ (US); Edward F. Diantonio, Staten Island, NY (US); Mark S. Felix, Uitikon (CH); Susan L. Jordan, Doylestown, PA (US)

(21) Appl. No.: 13/123,061

(22) PCT Filed: Oct. 6, 2009
(86) PCT No.: PCT/US09/59616

§ 371 Date: May 27, 2011

Related U.S. Application Data
(60) Provisional application No. 61/103,257, filed on Oct. 7, 2008.

(51) Int. Cl. A61K 8/92; A61K 8/41; A61K 8/29; A61K 8/27; A61Q 17/00; A61Q 5/02; A61Q 5/12; A61Q 5/06

(52) U.S. Cl. 424/70.9; 8/442

(57) ABSTRACT

Described are hair care compositions, comprising transparent inorganic metal oxide sunscreen particles and at least one of a cosmetically acceptable surfactant, conditioning agent, styling agent, humectant, emollient, preservative, or rheology modifier.

### CROSS REFERENCE TO RELATED APPLICATIONS

This Application claims the benefit of U.S. Provisional Application No. 61/103,257, filed on Oct. 7, 2008.

### FIELD

The present invention relates to compositions for use in hair conditioning and styling, and methods of preventing color fading due to exposure to sunlight (“sun-bleaching”) and damage to hair.

### BACKGROUND

The desirability of protecting hair from ultraviolet (UV) damage was recognized long ago. It is well know that hair can be damaged by the sun's rays, and, of course, colored hair in particular may experience detrimental effects on its color, shade, intensity, vibrancy, and/or luster. In the past, attempts to prevent such damage or fading were stymied by poor aesthetics. The success of a cosmetic, including hair care compositions, depends in great measure on the way it feels to a user at the time of use. For example, conventional sunscreens cannot simply be applied to hair, as they will not have acceptable aesthetics.

More recently, UV filters for hair based on organic chemical compositions have been attempted, but have not gained wide market acceptance based on their proclivity to wash off easily. While some newly developed quaternized UV filters for hair are far more efficacious at staying on hair, they are viewed with skepticism by some consumers in the wake of studies that have warned of oxybenzone use and health concerns.

Thus, what is needed are ways of protecting hair from UV damage using inert “mineral” UV filters having acceptable aesthetics.

### SUMMARY

In one embodiment, the present invention provides hair care compositions, comprising transparent inorganic metal oxide sunscreen particles and at least one of a cosmetically acceptable surfactant, conditioning agent, styling agent, humectant, emollient, preservative, or rheology modifier.

### DETAILED DESCRIPTION

In one embodiment, the present invention provides hair care compositions, comprising transparent inorganic metal oxide sunscreen particles and at least one of a cosmetically acceptable surfactant, conditioning agent, styling agent, humectant, emollient, preservative, or rheology modifier.

Most inorganic metal oxide sunscreen particles used in a sunscreen produce a cosmetically undesirable white appearance caused by light scattering. Moreover, the normal ingredients of a sunscreen composition are unsuitable for hair. Thus, the term “transparent inorganic metal oxide sunscreen particles,” as used herein has a special meaning, referring to inorganic metal oxide sunscreen particles produced by a variety of processing conditions which render the inorganic metal oxide compositions as clear, or transparent, upon application. In other words, these specially processed inorganic metal oxide compositions do not appear white once applied, hence the moniker of transparent.

Examples of transparent zinc oxide are disclosed in, for example, U.S. Pat. Nos. 5,223,250; 5,372,805; 5,573,753; 5,587,148; and 5,876,688. One example of a transparent zinc oxide is commercially available under the tradename Z-COTE from BASF Corporation (Germany). Another example of transparent zinc oxide is commercially available under the tradename ZINCLEAR IM from Antaria Limited (Australia). Another example of transparent zinc oxide is commercially available under the tradename Z-CLEAR from Actifirm (USA).

Examples of transparent titanium dioxide are disclosed in, for example, U.S. Pat. Nos. 5,573,753, 5,733,895, and 7,390,355. Examples of transparent titanium dioxide are commercially available under the tradenames TIPAQUE and TTO-51(A) from Ishihara Sangyo Kaisha, Ltd. (Japan). Another example of a transparent titanium dioxide is commercially available under the tradename T-COTE from BASF Corporation (Germany). Another example of transparent titanium dioxide is commercially available under the tradename UFTR from Miyoshi Kasei (Japan). Another example of transparent titanium dioxide is commercially available under the tradename SOLAVEIL CLARUS from Uniqema (Great Britain).

In one embodiment, the transparent inorganic metal oxide sunscreen particles are selected from transparent zinc oxide, titanium dioxide, or mixtures thereof.

In one embodiment, the transparent inorganic metal oxide sunscreen particles are present in an amount from about 0.1% to about 25% by weight of the composition, preferably from about 0.2% to about 10%, and more preferably 0.5% to about 5%. Armed with this knowledge, those skilled in the art can readily determine the amounts and combinations of additional ingredients to produce hair care compositions.

In one embodiment, the hair care composition of the present invention is a styling composition, for instance, a gel, a spray, or a mousse.

In one embodiment, the hair care composition of the present invention is a rinse off conditioner.

In one embodiment, the hair care composition of the present invention is a leave-in conditioner, for example a spray, foam, lotion, or pomade. Leave-in conditioners generally require lower concentrations of active ingredients, because unlike rinse-off conditioners, there is little danger of the active being rinsed down the drain. In one embodiment, the leave-in conditioner is formulated to reduce volume of frizzy hair. In one embodiment, the leave-in conditioner includes an oil to increase conditioning effect and produce a wet look aesthetic.

Compositions of the present invention can further incorporate other ingredients known in the art of hair care formulations including at least one of cosmetically acceptable emollients, moisturizers, conditioners, oils, suspending agents, opacifiers/pearlizers, sunscreens, surfactants, emulsifiers, preservatives, rheology modifiers, colorants, pH adjustors, propellants, reducing agents, anti-oxidants, fragrances, foaming or de-foaming agents, anti-static agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides. It is understood that there is no standard classification for ingredient types, and thus the following attempt to give examples for some categories is meant to be helpful, and not limiting. Certain ingredients, like monoquats, could reasonably be placed in several of the following categories, but the lists are meant to be exemplary, and not exhaustive.

Preferred surfactants include surface active materials formed from fatty alcohols, for example stearyl alcohol, cetyl alcohol, cetearyl alcohol and ceteareth-20, stearamidpropyl dimethylamine. Surfactants also include cosmetically acceptable emulsifiers. Preferably, the surfactant does not have detergency characteristics.

In one embodiment, the conditioning agent includes cationic monoquats, such as stearalkonium chloride, dicetyldimonium chloride, cetrimonium chloride, lauryl methyl gluceth-10 hydroxypropyldimonium chloride, and behenyltrimonium chloride and natural and synthetic cationic conditioning polymers such as polyquaternium-4, polyquaternium-7, polyquaternium-10, polyquaternium-24, polyquaternium-67 and the like, chitosan and derivatives thereof.

In one embodiment, the conditioning agent includes oils. Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), linolenic alcohol, oleyl alcohol, and the oil of cereal germs, synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, such as dimethylopolysiloxane and cyclomethicone, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Preferred styling agents include film formers and polymers. For example, when the hair composition is a gel, the styling agent includes at least one of polyvinyl pyrrolidone (PVP), acrylamide, acrylate, or polyvinyl pyrrolidone vinyl acrylic copolymer (PVP/VA). When the hair composition is a spray, the styling agent may be a urethane. When the hair composition is a mousse, the styling agent includes cellulosics.

In one embodiment, the humectant, sometimes described as a moisturizer, includes 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, and polyols, such as, for example, propylene glycol, glycerol and sorbitol, hydroxyalkyl urea. In one embodiment, the humectant is GLUCAM E-10 methyl gluceth-10.

In one embodiment, the emollient includes UCON FLUID PPG-14 Butyl Ether available from The Dow Chemical Company, dicaprylyl ether, C₁₂₋₁₅ alkyl benzoate, DC 200 FLUID 350 silicone fluid (from Dow Coming Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

Preferred preservatives include alcohols, aldehydes, methylchloroisothiazolinone and methylisothiazolinone, p-hydroxybenzoates, and in particular methylparaben, propylparaben, glutaraldehyde and ethyl alcohol. In one embodiment, the preservative is DMDM Hydantoin.

In one embodiment, the rheology modifier is a thickener, such as a cellulosic, gum, or other polymer.

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

The pH adjustors include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

In another embodiment, the present invention provides a method of preventing color fading of hair due to exposure to sunlight, comprising applying the hair care compositions of the present inventions to the hair. This is particularly desirable for protecting colored hair from detrimental effects on its color, shade, intensity, vibrancy, and/or luster.

### EXAMPLES

The following examples are for illustrative purposes only and are not intended to limit the scope of the present invention. All percentages are by weight unless otherwise specified.

### Example 1

Exemplary hair care compositions contain the components recited in TABLES 1A and 1B.

TABLE 1A

| | | Batch | Batch | Batch | Batch |
|---|---|---|---|---|---|
| | Component | 1 | 2 | 3 | 4 |
| A | PROMULGEN D cetearyl alcohol | 3.5 | 3.5 | 3.5 | 3.5 |
| | and ceteareth-20 | | | | |
| | DC 345 cyclomethicone | 1 | 1 | 1 | 1 |
| | JEENCHEM S-13 stearamidpropyl | 0.8 | 0.8 | 0.8 | 0.8 |
| | dimethylamine | | | | |
| | VARISOFT 432 PPG | 2 | 2 | 2 | 2 |
| | dicetyldimonium chloride | | | | |
| | stearyl alcohol | 1.5 | 1.5 | 1.5 | 1.5 |
| | ZINCLEAR S_60 AB (60% active) | 10 | 5 | — | — |
| | transparent zinc oxide | | | | |
| | ZINCLEAR IM_50 AB (50% | — | — | 10 | 6 |
| | active) transparent zinc oxide | | | | |
| B | Deionized Water | 79.8 | 84.8 | 79.8 | 83.8 |
| | GLUCAM E-10 methyl gluceth-10 | 1 | 1 | 1 | 1 |
| C | GLYDANT DMDM Hydantoin | 0.4 | 0.4 | 0.4 | 0.4 |

TABLE 1B

| | | Batch | Batch | Batch | Batch |
|---|---|---|---|---|---|
| | Component | 5 | 6 | 7 | 8 |
| A | PROMULGEN D cetearyl alcohol | 3.5 | 3.5 | 3.5 | 3.5 |
| | and ceteareth-20 | | | | |
| | DC 345 cyclomethicone | 1 | 1 | 1 | 1 |
| | JEENCHEM S-13 stearamidpropyl | 0.8 | 0.8 | 0.8 | 0.8 |
| | dimethylamine | | | | |
| | VARISOFT 432 PPG | 2 | 2 | 2 | 2 |
| | dicetyldimonium chloride | | | | |
| | stearyl alcohol | 1.5 | 1.5 | 1.5 | 1.5 |
| | ZINCLEAR IM_50 CCT (50% | 10 | 6 | — | — |
| | active) transparent zinc oxide | | | | |
| | Z-COTE MAX transparent zinc | — | — | 6 | — |
| | oxide (50 wt. % suspension in | | | | |
| | CETIOL AB C₁₂₋₁₅ alkyl benzoate) | | | | |
| | TTO 51 transparent titanium | — | — | — | 3 |
| | dioxide | | | | |
| B | Deionized Water | 79.8 | 83.8 | 83.8 | 86.8 |
| | GLUCAM E-10 methyl gluceth-10 | 1 | 1 | 1 | 1 |
| C | GLYDANT DMDM Hydantoin | 0.4 | 0.4 | 0.4 | 0.4 |

Amounts are expressed in wt. % (percent of individual component weight as compared to formulation weight). The transparent inorganic metal oxides were ZINCLEAR-S_{—}60AB: siliconate-coated ZnO 60 wt. % dispersion in C12-C15 alkyl benzoate from Antaria; ZINCLEAR IM 50AB micron-sized ZnO 50 wt. % dispersion in C12-C15 alkyl benzoate from Antaria; ZINCLEAR IM_{—}50CCT: micron-sized ZnO 50 wt. % dispersion in caprylic/capric triglycerides from Antaria; TTO-51(A) alumina-treated ultrafine TiO₂ from Ishihara Sangyo Kaisha, Ltd. (Tokyo 03-3230-8623, Japan) with average particle size of 0.03˜0.05 microns; and Z-Cote® MAX™ silicone treated USP grade of ZnO with average particle size≦0.200 microns.

These formulations are particularly suitable as rinse-off conditioners.

Phase A ingredients are combined, mixed, and heated to about 75° C. with stirring. Phase B ingredients are combined, mixed, and heated to about 75° C. with stirring in a separate vessel. Phase B is added to Phase A at about 75° C. under stirring. The mixture is allowed to cool to ambient temperature with stirring, and then Phase C is added at about 35° C. If needed, the pH of the formulation is adjusted to slightly acidic or neutral (6-7) using citric acid.

### Example 2 (Comparative)

Comparative compositions contain the components recited in TABLE 2.

TABLE 2

| | | Comparative | Comparative |
|---|---|---|---|
| | Component | Batch A | Batch B |
| A | PROMULGEN D cetearyl alcohol | 3.5 | 3.5 |
| | and ceteareth-20 | | |
| | DC 345 cyclomethicone | 1 | 1 |
| | JEENCHEM S-13 stearamidpropyl | 0.8 | 0.8 |
| | dimethylamine | | |
| | VARISOFT 432 PPG | 2 | 2 |
| | dicetyldimonium chloride | | |
| | stearyl alcohol | 1.5 | 1.5 |
| | SOLAMER GR8 (72% active) | 4.2 | — |
| | polyamide-2 (UV filter) | | |
| B | Deionized Water | 85.6 | 89.8 |
| | GLUCAM E-10 methyl gluceth-10 | 1 | 1 |
| C | GLYDANT DMDM Hydantoin | 0.4 | 0.4 |

Amounts are expressed in wt. %. Batches are prepared as described in Example 1.

### Example 3

Eight inch long hair tresses (˜4 g, flat swatches) of European virgin medium brown hair (International Hair Importers and Products Inc., Glendale, N.Y.) were pre-washed and pre-hydrated, and then an individual tress was treated with 0.5 g of one formulation selected from Batch 1 (prepared substantially according to the protocol of Example 1) and Comparative Batch B (prepared substantially according to the protocol of Example 2). The selected formulation was worked into the hair for about 1 min and then rinsed off under running tap water at about 38° C. and about 0.4 gal/min for about 1 min. At the end of the rinsing cycle, the excess of water was squeezed out and the treatment was repeated one more time with another 0.5 g of the same selected formulation. After two consecutive washings as described above, hair tresses were hung to dry overnight.

Upon drying, both tresses (i.e., one treated with Batch 1 and one treated with Comparative Batch B) felt soft and clean and had no white residue.

Scanning electron microscopy (SEM; Hitachi S-4500 Field Emission Scanning Electron Microscope with accelerating voltage of 3 kv) was used to analyze the tresses. Comparative Batch B had no deposition as determined by SEM. Batch 1 showed extensive deposition.

Using energy dispersive x-ray spectroscopy (EDS) techniques, the surface morphology of hair fibers/deposition was assessed. Back scattered detector fixture, which is capable of highlighting the contrast between elements with different atomic weights such as, for example, C and Zn was used to estimate the amount of zinc (from the zinc oxide) deposited on hair fibers. Images obtained showed that about 50% of the hair fiber surface treated with Batch 1 was coated with Zn or ZnO. Comparative Batch B had no deposition.

These findings demonstrated the ability of small particle size ZnO to deposit on hair from rinse-off formulations. As noted above, the aesthetics of the inventive hair tresses were found acceptable and similar to the comparative tresses.

### Example 4

Tresses were prepared and treated as described in Example 3, with the formulations described below prepared substantially according to the protocols of Examples 1 and 2.

None of the hair tresses had any white residue. Batch 5 (5 wt. % ZnO dispersion in caprylic/capric triglycerides (CCT)) imparted hair tresses with greasy/heavy feel. This negative impact on hair aesthetics was significantly diminished in the case of Batch 6, which contained only 1 wt. % of ZnO CCT dispersion. No negative impact on hair aesthetics was observed for Batches 3 and 4 (ZnO in C12-C15 alkyl benzoate (AB)).

As a means of quantifying the zinc (from the zinc oxide) deposited on hair tresses, Induction-Coupled Plasma Optical Emission Spectroscopy (ICP-OES) studies were undertaken. The tresses were ashed in a programmable furnace. The residue ash was dissolved using nitric and hydrofluoric acids (the Savillex Teflon digestion method). The aqueous solutions were analyzed on a PerkinElmer Optima DV 4300 model ICP spectrometer equipped with a corrosion-resistant sample introduction system. A general ICP method for trace metal analysis against elemental calibration curves created from certified ICP standards was used. A known standard was analyzed together with the hair samples for recovery and accuracy control. Samples were run in duplicates. Hair was cut once from the tip of the tress and once from the top part close to the swatching. The results are shown in TABLE 3.

TABLE 3

| | Batch | Batch | Batch | Batch | Comparative |
|---|---|---|---|---|---|
| | 3 | 4 | 5 | 6 | Batch B |
| Zinc, ppm | 4250 | 3250 | 5000 | 2000 | 170 |

As shown in TABLE 3, significant amounts of zinc were detected on hair treated with conditioners of the invention, Batches 3-6.

### Example 5

Tresses were prepared and treated as described in Example 3, with the formulations below prepared substantially according to the protocols of Examples 1 and 2.

None of the hair tresses had any white residue. As a means of quantifying the titanium (from the titanium dioxide) deposited on hair tresses, Induction-Coupled Plasma Optical Emission Spectroscopy (ICP-OES) studies were undertaken as described in Example 4. The results are shown in TABLE 4.

TABLE 4

| | Batch | Comparative |
|---|---|---|
| | 8 | Batch B |
| Titanium, ppm | 433 | 0.9 |

As shown in TABLE 4, significant amounts of titanium were detected on hair treated with the conditioners of the invention, Batch 8.

### Example 6

Eight inch long hair tresses (˜4 g, flat swatches) were prepared. The hair was European single-bleached hair (International Hair Importers and Products Inc., Glendale, N.Y.) which was dyed with a commercial oxidative dye product (Cherry Cordial 6.6 (Bright Auburn) COULEUR EXPERTE from L'Oreal). Deep red coloration was achieved by following the product application instructions.

The colored hair tresses were pre-washed and pre-hydrated, and then an individual tress was treated with 0.3 g of one of the formulations listed below prepared substantially according to the protocols described in Examples 1 and 2. The selected formulation was worked into the hair for about 1 min and then rinsed off under running tap water at about 38° C. and about 0.4 gal/min for about 1 min. At the end of the rinsing cycle, the excess of water was squeezed out and the treatment was repeated one more time with another 0.5 g of the same selected formulation. After two consecutive washings as described above, hair tresses were hung to dry overnight. All tresses looked the same; no difference in coloration could be perceived by naked eye.

Sunlight exposure was simulated using an Atlas Ci-65A WEATHEROMETER equipped with water cooled xenon burner, type S-Borosilicate inner and outer filters, and sample holder specifically designed to fit on the Ci-65 sample rack. The following settings were used during the study:

Program 0—All light, no water spray

Irradiance—0.35 W/m²@340 nm

Black panel temperature—55° C.

Dry bulb temperature—36° C.

Wet bulb temp—25° C.

Relative Humidity—41%

After 4 days, the tresses were taken out and an observation was made that all tresses looked “faded” compared to a hair tress that was set aside and had not been exposed to xenon light. In order to assess the degree of fading among the irradiated samples, a series of panel studies were conducted.

Ten expert panelists trained to evaluate performance of cosmetic products on hair were asked to evaluate the intensity of coloration of hair tresses. Each panelist evaluated a pair of tresses, one tress treated with a composition of the invention versus one treated with control. The panelists were asked to pick one tress that had more intense/deep coloration. Additionally, the subjective evaluations were statistically analyzed to identify differences at above 89% confidence level. The following keys were used:

+ the experimental sample was statistically superior to the control (minimum 8 out of 10 times the experimental sample was rated superior);

= the experimental sample showed no statistical difference to the control (3-7 of 10 times the experimental sample was picked over the control; and

− the experimental sample was statistically inferior to the control (the experimental sample was picked over the control not more than 2 out of 10 times).

The results are indicated in TABLE 5.

TABLE 5

| | Color Intensity |
|---|---|
| Batch 4 versus Comparative Batch A | 8/10 (+) |
| Batch 4 versus Comparative Batch B | 10/10 (+) |
| Batch 8 versus Comparative Batch B | 8/10 (+) |

Eight out of ten panelists found the color of hair tresses treated with a formulation of the invention (Batch 4) to be more intense and deep compared to control tresses treated with the formulation containing the SOLAMER GR8 commercial UV filter for hair products (Comparative Batch A). In other words, Batch 4 prevented color fading. The result was statistically significant.

All panelists found the color of hair tresses treated with a formulation of the invention (Batch 4) to be more intense and deep compared to blank control (Comparative Batch B). The result was statistically significant.

Eight out of ten panelists found the color of hair tresses treated with a formulation of the invention (Batch 8) to be more intense and deep compared to blank control (Comparative Batch B). The result was statistically significant.

### Example 7

Eight inch long hair tresses (˜4 g, flat swatches) were prepared. The hair was obtained from International Hair Importers and Products Inc., Glendale, N.Y., as either European virgin medium brown hair or European single-bleached hair. If the latter, it was dyed with a commercial oxidative dye product (Cherry Cordial 6.6 (Bright Auburn) COULEUR EXPERTE from L'Oreal) to yield a colored hair as described above. Deep red coloration was achieved by following the product application instructions.

The European virgin medium brown or colored hair tresses were pre-washed and pre-hydrated, and then an individual tress was treated with 0.3 g of one of the formulations below prepared substantially according to the protocols described in Examples 1 and 2. The selected formulation was worked into the hair for about 1 min and then rinsed off under running tap water at about 38° C. and about 0.4 gal/min for about 1 min. At the end of the rinsing cycle, the excess of water was squeezed out and the treatment was repeated one more time with another 0.5 g of the same selected formulation. After two consecutive washings as described above, hair tresses were hung to dry overnight. All tresses were visually inspected and found to have good aesthetics without any white residue.

Induction-Coupled Plasma Optical Emission Spectroscopy (ICP-OES) studies were undertaken as described in Example 4. The results are shown in TABLE 6.

TABLE 6

| | Batch | Batch | Comparative |
|---|---|---|---|
| | 1 | 2 | Batch B |
| European Virgin Brown Hair | 1750 | 930 | 295 |
| Zinc, ppm | | | |
| Colored Hair | 2300 | 1200 | 340 |
| Zinc, ppm | | | |

Significant amounts of zinc were detected on hair treated with the inventive conditioners, confirming that zinc oxide had been deposited. The amount of deposition appears to correlate with ZnO concentration, in that Batch 2 has approximately half the zinc concentration of Batch 1.

The amount of elemental Zn detected on colored hair was higher compared to virgin brown hair.

### Example 8

Exemplary leave-in hair care compositions contain the components in TABLE 7.

TABLE 7

| | Batch | Batch | Batch |
|---|---|---|---|
| Component | 9 | 10 | 11 |
| Cetyl alcohol | 2.0 | 2.0 | 2.0 |
| Stearyl alcohol | 3.0 | 3.0 | 3.0 |
| PROQUAT CT 29 (29% active) | 3.4 | 3.4 | 3.4 |
| cetrimonium chloride | | | |
| CELLOSIZE PCG-10 | 0.5 | 0.5 | 0.5 |
| hydroxyethylcellulose | | | |
| Hydrogenated vegetable oil | 2.0 | 2.0 | 2.0 |
| Panthenol | 0.1 | 0.1 | 0.1 |
| ZINCLEAR IM_50 AB (50% | 1.0 | 2.0 | 4.0 |
| active) transparent zinc oxide | | | |
| GLYDANT DMDM Hydantoin | 0.4 | 0.4 | 0.4 |
| Deionized Water | 87.6 | 86.6 | 84.6 |

Amounts are expressed in wt. %. To prepare the formulations, hydroxyethylcellulose is sprinkled into rapidly agitated room temperature water. Once uniform, the solution is heated to 60° C. and cetrimonium chloride is added under stirring. The solution is then heated to 75-80° C. In a separate container, fatty alcohols are heated to 75-80° C. and mixed until uniform. This mixture is added to the previously described hydroxyethylcellulose containing solution and mixed until uniform. The resulting batch is cooled below 40° C. and vegetable oil and panthenol are added, followed by the ZINCLEAR suspension. The pH is adjusted to 6-7 with citric acid, and the batch cooled to room temperature, after which, hydantoin is added and QS water.

### Example 9 (Comparative)

Comparative compositions contain the components recited in TABLE 8.

TABLE 8

| | Comparative | Comparative |
|---|---|---|
| Component | Batch C | Batch D |
| Cetyl alcohol | 2.0 | 2.0 |
| Stearyl alcohol | 3.0 | 3.0 |
| PROQUAT CT 29 (29% active) | 3.4 | 3.4 |
| cetrimonium chloride | | |
| CELLOSIZE PCG-10 | 0.5 | 0.5 |
| hydroxyethylcellulose | | |
| Hydrogenated vegetable oil | 2.0 | 2.0 |
| Panthenol | 0.1 | 0.1 |
| GLYDANT DMDM Hydantoin | 0.4 | 0.4 |
| CETIOL AB C12-15 alkyl benzoate | — | 1.0 |
| Deionized Water | 88.6 | 87.6 |

Amounts are expressed in wt. %. Batches are prepared as described in Example 8.

### Example 10

Pre-washed and pre-hydrated eight inch long commercial frizzy hair tresses (˜4.5 g, round swatching) were treated with 0.45 g of conditioner product each prepared substantially according to the protocols described in Examples 8 and 9. Conditioner product was worked into the hair for 0.5 min on one side and then for 0.5 min on the other side. After that the hair tresses were hung to dry overnight.

The inspection of hair tresses treated with Batches 9-11 in comparison to similar tresses treated with comparative Batches C and D revealed no white residue and confirmed acceptable, and overall comparable, aesthetics of all tresses.

It is understood that the present invention is not limited to the embodiments specifically disclosed and exemplified herein. Various modifications of the invention will be apparent to those skilled in the art. Such changes and modifications may be made without departing from the scope of the appended claims. Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A hair care composition, comprising:
transparent inorganic metal oxide sunscreen particles; and
at least one of a cosmetically acceptable surfactant, conditioning agent, styling agent, humectant, emollient, preservative, or rheology modifier.

2. The hair care composition of claim 1, wherein the transparent inorganic metal oxide sunscreen particles are selected from transparent zinc oxide, titanium dioxide, or mixtures thereof.

3. The hair care composition of claim 1, wherein the transparent inorganic metal oxide sunscreen particles are present in an amount from about 0.1% to about 25% by weight of the composition, preferably from about 0.2% to about 10%, and more preferably 0.5% to about 5%.

4. The hair care composition of claim 1, wherein the hair care composition is a conditioner.

5. The hair care composition of claim 4, wherein the conditioner is a rinse-off conditioner.

6. The hair care composition of claim 5, wherein the rinse-off conditioner includes behenyltrimonium chloride.

7. The hair care composition of claim 6, wherein the PPG dicetyldimonium chloride is present in an amount from about 0.1% to about 10% by weight of the composition, preferably from about 1% to about 3%, and more preferably about 2%.

8. The hair care composition of claim 4, wherein the conditioner is a leave-in conditioner.

9. The hair care composition of claim 4, wherein the conditioner includes at least one of stearyl alcohol or cetyl alcohol.

10. The hair care composition of claim 1, wherein the hair care composition is a shampoo, non-conditioning rinse, non-conditioning gel, styling/fixative product, or hair gel.

11. The hair care composition of claim 1, further comprising at least one colorant, preservative, pH adjustor, propellant, reducing agent, fragrance, foaming agent, flavor, astringent, antiseptic, insect repellant, bleach, lightener, anti-dandruff agent, strengthener, filler, barrier material, or biocide.

12. A method of preventing color fading of hair due to exposure to sunlight, comprising: applying the hair care composition of claim 1 to the hair.

13. The method of claim 12, wherein the hair has been previously treated with an oxidative dye.
